(19) United States
(12) Patent Application Publication
(10) Pub. No.: US 2008/0167673 A1
(43) Pub. Date: Jul. 10, 2008

(54) Lancet Release Mechanism

(76) Inventors: Weiping Zhong, Granger, IN (US); Tieming Ruan, Granger, IN (US); Kevin Chang, Granger, IN (US)

Correspondence Address: NIXON PEABODY LLP, 161 N. CLARK STREET, 48TH FLOOR, CHICAGO, IL, 60601, US

(21) Appl. No.: 11/885,527

(22) PCT Filed: Mar. 3, 2006
(86) PCT No.: PCT/US06/07678

§ 371 Date: Aug. 31, 2007

Related U.S. Application Data
(60) Provisional application No. 60/658,844, filed on Mar. 4, 2005.

(51) Int. Cl. A61B 5/151

(52) U.S. Cl. 606/181

(57) ABSTRACT

An end cap is removably attachable to a lancing device and comprises an end cap, a plurality of release buttons, and a plurality of engagement mechanisms. The end cap has an external surface. The plurality of release buttons is accessible from external the end cap housing. Each of the plurality of engagement mechanisms is attached to one of the plurality of release buttons. The plurality of engagement mechanisms is adapted to engage a lancet assembly. The depression of the plurality of release buttons towards the external surface of the end cap housing causes the plurality of engagement mechanisms to engage the lancet assembly.

### FIELD OF THE INVENTION

The present invention relates generally to diagnostic instruments and, more particularly, to a lancet-release mechanism for a lancing device.

### BACKGROUND OF THE INVENTION

The quantitative determination of analytes in body fluids is of great importance in the diagnoses and maintenance of certain physiological abnormalities. For example, lactate, cholesterol and bilirubin should be monitored in certain individuals. In particular, determining glucose in body fluids is important to diabetic individuals who must frequently check their blood glucose levels to regulate the glucose intake in their diets.

One method of obtaining a body fluid sample, such as a whole blood sample, is to use a lancing device. The whole blood sample may then be used to determine the glucose concentration of an individual. Existing lancing devices use a lancet to pierce the tissue of the skin, allowing a blood sample to form on the skin's surface. Typically, lancing devices hold the lancet within them when the lancet is not in use, so as to shield the user from injury as well as to assist in preventing or inhibiting contamination.

Existing lancing devices require two-handed operation, are dangerous, or are ineffective in releasing the lancet. Two-handed operation requires that one hand hold the lancing device while the other hand removes the lancet. This is inconvenient to many users as the lancet is small, and may cause safety problems as the lancet could pierce the user's skin inadvertently. This can cause the user pain and may also transmit diseases. Some one-handed designs eject the lancet too hard such that ejecting the lancet becomes dangerous if the lancet is not re-shielded. Still other one-handed designs do not eject the lancet effectively, as the lancet is not released from the lancet holder even after the user depresses the release mechanism. Yet other one-handed designs have complicated release mechanisms internally, such that if a user drops the lancing device, the release mechanism may jam and no longer eject the lancet from the lancing device.

It would be desirable to have a lancing device and a method for using a lancing device that addresses these issues.

### SUMMARY OF THE INVENTION

According to one embodiment of the present invention, an end cap that is removably attachable to a lancing device is disclosed. The end cap comprises an end cap, a plurality of release buttons, and a plurality of engagement mechanisms. The end cap has an external surface and the plurality of release buttons are accessible from external the end cap housing. Each of the plurality of engagement mechanisms are attached to one of the plurality of release buttons. The plurality of engagement mechanisms are adapted to engage a lancet assembly. The depression of the plurality of release buttons towards the external surface of the end cap housing causes the plurality of engagement mechanisms to engage the lancet assembly.

According to another embodiment of the present invention, a lancing device is disclosed. The lancing device comprises a main housing, a lancet holder, and an end cap. The lancet holder is located within the main housing and forms an aperture. The aperture is adapted to receive a lancet assembly therein. The end cap is adapted to be removably attached to the main housing. The end cap includes a plurality of release buttons accessible from external the end cap. Each of the plurality of release buttons is attached to an engagement mechanism adapted to engage the lancet assembly. The depression of the plurality of release buttons causes the plurality of engagement mechanisms to engage the lancet assembly such that removal of the end cap from the main housing removes the lancet assembly from the lancet holder.

According to yet another embodiment of the present invention, a method for removing a lancet assembly from a lancet holder is disclosed. The method comprises the acts of providing a lancing device and an end cap as described above. At least one of the plurality of release buttons is depressed in the direction of the lancet assembly. The lancet assembly is engaged with the plurality of engagement mechanisms when at least one of the plurality of release buttons is depressed. The end cap is detached from the main housing and the lancet assembly is removed from the lancet holder as the end cap is detached.

The above summary of the present invention is not intended to represent each embodiment, or every aspect, of the present invention. Additional features and benefits of the present invention are apparent from the detailed description and figures set forth below.

### BRIEF DESCRIPTION OF THE DRAWINGS

FIG. 1a is a top view of a lancing device, according to one embodiment of the present invention.

FIG. 1b is a bottom view of the lancing device of FIG. 1a.

FIG. 2 is a side view of the lancing device of FIGS. 1a-b.

FIG. 3 is a cross-sectional, top view of the lancing device of FIG. 2 in a resting position.

FIG. 4 is a cross-sectional, top view of the lancing device of FIG. 2 in a cocking position.

FIG. 5 is a perspective view of a lancing mechanism, according to one embodiment of the present invention.

FIG. 6a is a side view of an end cap, in its resting position, adapted to be removably attached to the lancing device of FIGS. 1a-b, according to one embodiment of the present invention.

FIG. 6b is a side view of the end cap of FIG. 6a in its release position.

FIG. 7 is a perspective view of a lancet assembly, according to one embodiment of the present invention.

FIG. 8a is a schematic illustration of the end cap of FIG. 6a removably attached to the lancing device having a lancet assembly in a cocked position.

FIG. 8b is a schematic illustration of the end cap of FIG. 6a with the lancet assembly in a resting position.

FIG. 8c is a schematic illustration of the end cap of FIG. 6b with the lancet assembly in the resting position.

FIG. 8d is a schematic illustration of the end cap of FIG. 6b after the end cap has been removed from the lancing device.

FIG. 8e is a schematic illustration of the end cap of FIG. 6a after the lancet assembly has been disengaged from the engagement mechanisms.

FIG. 9a is a side view of an end cap, in its resting position, adapted to be removably attached to the lancing device of FIGS. 1a-b, according to another embodiment of the present invention.

FIG. 9b is a side view of the end cap of FIG. 9a in its release position.

FIG. 10 is a perspective view of a lancet assembly, according to another embodiment of the present invention.

### DESCRIPTION OF ILLUSTRATED EMBODIMENTS

The present invention is directed to a lancet-release mechanism for incorporation into a stand-alone lancing device or into a lancing device that is incorporated into a meter or similar testing device. The lancing device is adapted to receive a lancet for use in drawing a body fluid from a test subject. The body fluid generally contains at least one analyte that may then be examined to determine its concentration in the body fluid sample.

Lancing devices and lancets may be used to produce a blood or body fluid sample from a test subject. This sample may then be analyzed with a meter and test strip, or similar devices, to determine the concentration of the analyte to be examined. Examples of the types of analytes that may be collected with a lancing device include glucose, lipid profiles (e.g., cholesterol, triglycerides, LDL and HDL), microalbumin, hemoglobin A1C, fructose, lactate, or bilirubin.

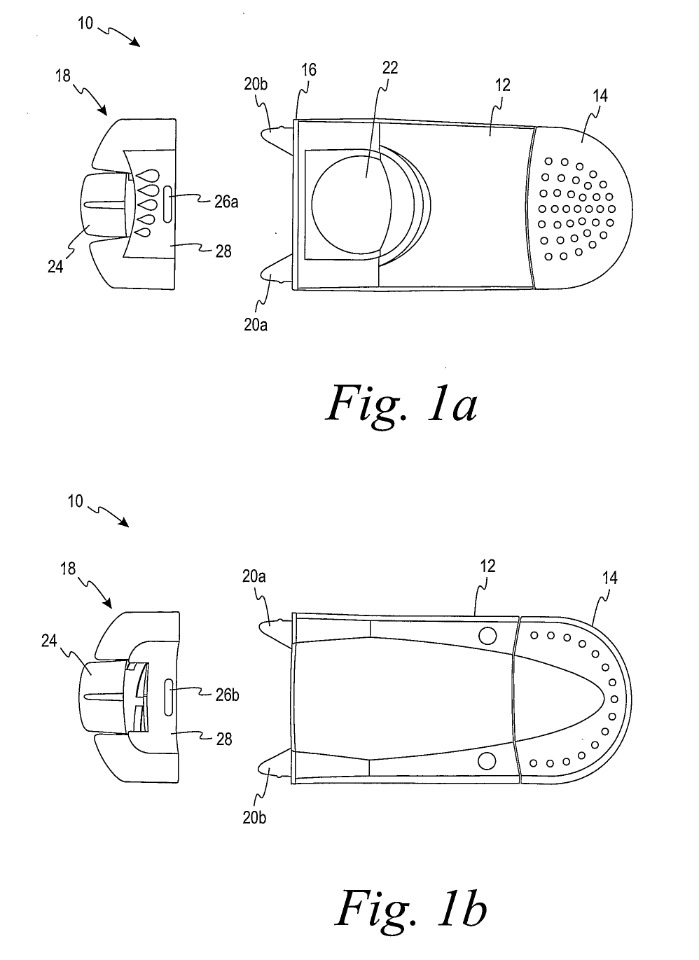

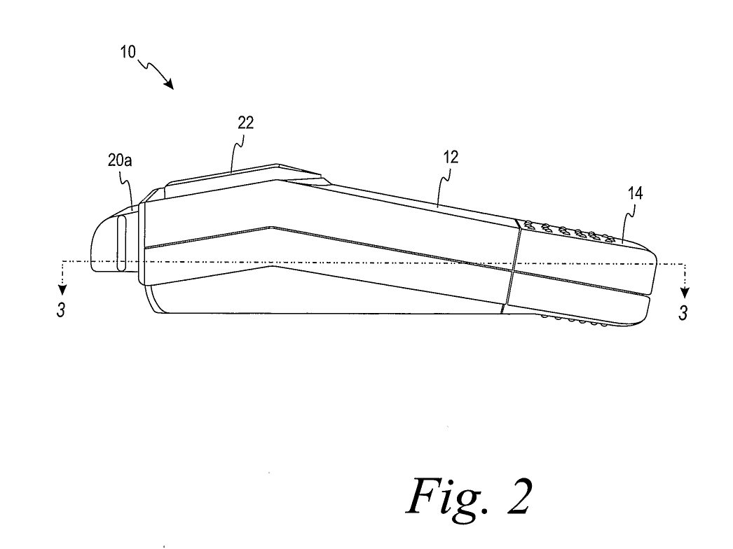

Tuning now to the drawings and initially to FIGS. 1-2, a lancing device 10 for obtaining a fluid sample from a test subject is illustrated, according to one embodiment of the present invention. The lancing device 10 has a main housing 12 and a movable housing 14 that is movable relative to the main housing 12. An end cap support 16 is connected to the main housing 12 on the testing end of the lancing device 10.

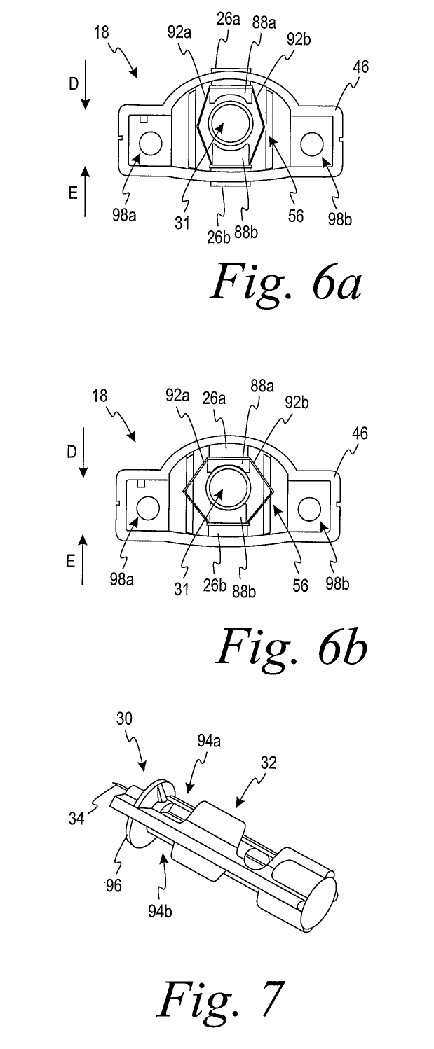

An end cap 18 may be removably attached to the end cap support 16. When attached, the end cap 18 is retained on the end cap support 16 by a pair of support arms 20a-b integrally formed with the end cap support 16. The end cap 18 includes a plurality of release buttons 26a-b that extend through apertures (not shown) in a housing 46 (FIGS. 6a-b). The release buttons 26a-b extend from an external surface 28 of the end cap 18 such that a user can engage and depress the release buttons 26a-b towards the external surface 28.

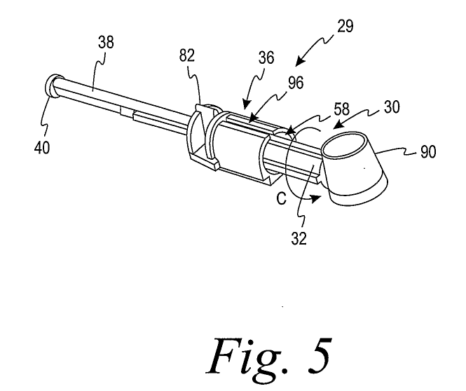

To use the lancing device 10, the movable housing 14 is pulled away from the main housing 12 to move an internal lancing mechanism 29 (as best illustrated in FIG. 5) to a cocked position, and then a pushbutton 22 is pushed to actuate the lancing mechanism 29 so that the sharp tip of a lancet is forced through an aperture 31 (FIGS. 6a-b) in the end cap 18. The lancing device 10 may be provided with a number of different end caps 18, each having a different width, to facilitate the formation of skin punctures of various depths. Alternatively, the end cap 18 may include an adjustable dial 24 for allowing punctures of different depths to be performed utilizing a single end cap 18.

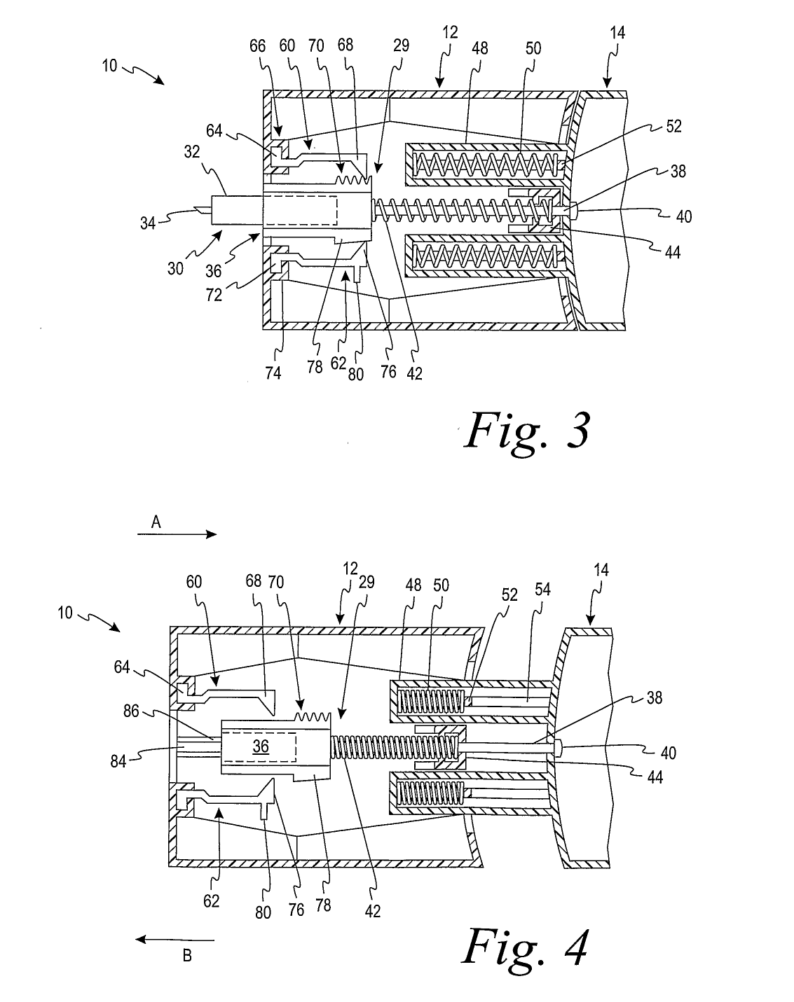

Turning now to FIG. 3, a cross-sectional view of a portion of the lancing device 10 is illustrated with the end cap 18 and end cap support 16 not shown. A lancet assembly 30 having a lancet body 32 and a lance 34 is received within an internal cylindrical aperture 58 (FIG. 5) formed in a generally cup-shaped lancet holder 36. The lancet holder 36 is connected to an elongated shaft 38 by being integrally formed therewith. The shaft 38 has an enlarged end 40 that is supported within the movable housing 14. A drive spring 42 is disposed around the shaft 38 between the lancet holder 36 and a spring stop 44 (see also FIG. 4) integrally formed with the main housing 12.

The movable housing 14 has a pair of elongate spring trays 48 integrally formed therewith. A return spring 50 is disposed within each of the spring trays 48, a first end of each return spring 50 being disposed against an internal surface of the spring tray 48 and a second end of each return spring 50 being disposed against a spring stop 52 integrally formed with the main housing 12. The spring stops 52 extend into the spring trays 48 through an elongate slot 54 (see FIG. 4) formed in the bottom portion of each tray 48.

A damping arm 60 and a retaining aim 62 are disposed adjacent opposite sides of the lancet holder 36. The damping arm 60 has a first end 64 that is held within a retaining structure 66 integrally formed with the main housing 12. A second pointed end 68 of the damping arm 60 is disposed adjacent a corrugated surface 70 formed on an outside portion of the lancet holder 36. The retaining arm 62 has a first end 72 that is held within a retaining structure 74 integrally formed with the main housing 12. A second pointed end 76 of the retaining arm 62 is disposed adjacent an angled stop member 78. The lower side of the retaining aim 62 rests on a support member 80. The arms 60, 62 are biased inwardly towards the lancet holder 36 so that they make contact with the respective outer sides of the lancet holder 36.

FIG. 3 illustrates the interior of the lancing device 10 when the lancing device 10 is not in use. In this position, the lancet holder 36 is disposed in a resting position between a puncture position and a cocked position. FIG. 4 illustrates the interior of the lancing device 10 (the lancet assembly 30 is not shown) when the lancet holder 36 is in a cocked position in which the movable housing 14 has been pulled away from the main housing 12.

Referring to FIG. 4, to move the lancet holder 36 from its resting position to its cocked position, the movable housing 14 is pulled away from the main housing 12 in the direction of Arrow A. The movable housing 14 continues to be pulled—against the force of the drive spring 42—until the angled stop member 78 formed on the lancet holder 36 moves past (to the right of as illustrated in FIG. 4) the pointed end 76 of the retaining arm 62. At that point, the bias of the retaining arm 62 will force its pointed end 76 inwardly, so that the pointed end 76 makes contact with the side of the lancet holder 36 disposed on the testing end side of the angled stop member 78. When in that cocked position, movement of the lancet holder 36 in the direction of Arrow B due to the drive spring 42 is prevented because of the contact between the pointed end 76 of the retaining arm 62 and the angled stop member 78. After the lancet holder 36 is placed in the cocked position, the user allows the return springs 50 to force the movable housing 14 back to its initial position adjacent the main housing 12.

The lancet holder 36 is guided between its resting and cocked positions by a guide rib 82 (FIG. 5) formed on the bottom portion of the lancet holder 36 that rides within a groove 84 formed between a pair of raised guide rails 86 formed in a bottom interior portion of the main housing 12.

To perform a puncture on a test subject's skin, the end cap 18 is attached to the lancing device 10. The lancet holder 36 may be in the cocked position at the time the end cap 18 is attached or may be cocked once the end cap 18 is in position. The end cap 18 is then placed firmly against the skin where the puncture is to be made, and the pushbutton 22 is depressed. Depressing the pushbutton 22 causes an angled release aim (not shown), integrally formed with the bottom of the pushbutton 22 and which passes through an aperture (not shown) in the main housing 12, to force the retaining arm 62 away from the lancet holder 36. Thus, the lancet holder 36 is no longer prevented from moving in the direction of Arrow B by the contact of the angled stop member 78 with the pointed end 76 of the retaining arm 62. A spring mechanism—for example, an elastically deformable foam material—may be disposed between the pushbutton 22 and a portion of the main housing 12 to bias the pushbutton 22 to its non-actuated position.

Upon release of the lancet holder 36 as described above, the drive spring 42 will force the lancet holder 36 in the direction of Arrow B until the sharp point of the lance 34 (FIG. 3) passes through the aperture (not shown) in the end cap 18 to make the puncture. When the puncture is made, the drive spring 42 will be in a stretched position, and after the puncture is made the contraction of the drive spring 42 will draw the lancet holder 36 back towards its resting position shown in FIG. 3.

As the lancet holder 36 moves from its puncture position back to its resting position shown in FIG. 3, the pointed tip 68 of the damping arm 60 will make frictional contact with the corrugated surface 70. This frictional contact decelerates or dampens the movement of the lancet holder 36. Such damping assists in preventing or inhibiting the drive spring 42—and its natural tendency to oscillate (due to its being elastically deformable)—from causing a second, unintended skin puncture to be made. As used herein, the term “corrugated” refers to a surface having raised ribs or other structures, either regularly or irregularly spaced, for providing an increased amount of function when the surface is brought into contact with a damping member.

Turning now to FIG. 5, a perspective view of the lancing mechanism 29 is illustrated, according to one embodiment of the present invention. The lancet mechanism 29 includes the lancet holder 36 adapted to receive the lancet assembly 30. As illustrated in FIG. 5, the lancet assembly 30 is disposed within the generally cylindrical aperture formed in the lancet holder 36. The lancet assembly 30 is shown with a protective cap 90 that has a portion that is integrally formed with the lancet body 32 and which covers the sharp point of the lance 34. Prior to using the lancing device 10, the lancet body 32 of a new lancet assembly 30 is inserted into the cylindrical aperture disposed in the lancet holder 36, and then the protective cap 90 is twisted off of the lancet assembly 30, in the direction of the Arrow C shown in FIG. 5.

Turning now to FIGS. 6a-b, the end cap 18 is illustrated as viewed from the attachable lancing device 10 (FIGS. 1-4). The end cap 18 includes attachment areas 98a-b adapted to removably attach the end cap 18 to the support arms 20a-b of the lancing device 10. The end cap 18 includes a lancet-release mechanism 56 adapted to assist in removing the lancet assembly 30 from the lancet holder 36. The lancet-release mechanism 56 includes the release buttons 26a-b, a plurality of engagement mechanisms 88a-b, and a plurality of springs 92a-b. Each of the release buttons 26a-b extends through an aperture (not shown) formed in the end cap housing 46. The release buttons 26a-b are each attached to one of the engagement mechanism 88a-b, respectively. The engagement mechanisms 88a-b are adapted to engage the lancet assembly 30 and can be used to remove the lancet assembly 30 from the lancet holder 36.

The engagement mechanisms 88a-b are attached via open-coiled, helical compression springs 92a-b. The springs 92a-b are positioned so as to bias the engagement mechanisms 88a-b and the release buttons 26a-b to their resting position, as illustrated in FIG. 6a. In this position, the engagement mechanisms 88a-b do not engage the lancet assembly 30 and the release buttons 26a-b extend from the end cap 18.

To engage the lancet assembly 30—to remove it from the lancet holder 36—the release buttons 26a-b are depressed in the direction of the lancet assembly 30, illustrated by Arrows D and E in FIGS. 6a-b. The release buttons 26a-b may be depressed until the ends are flush with the external surface 28 (FIGS. 1a-b) of the end cap 18. The depression of the release buttons 26a-b move the lancet-release mechanism to the release position as illustrated in FIG. 6b, causing the springs 92a-b to flex and the engagement mechanisms 88a-b to move towards each other.

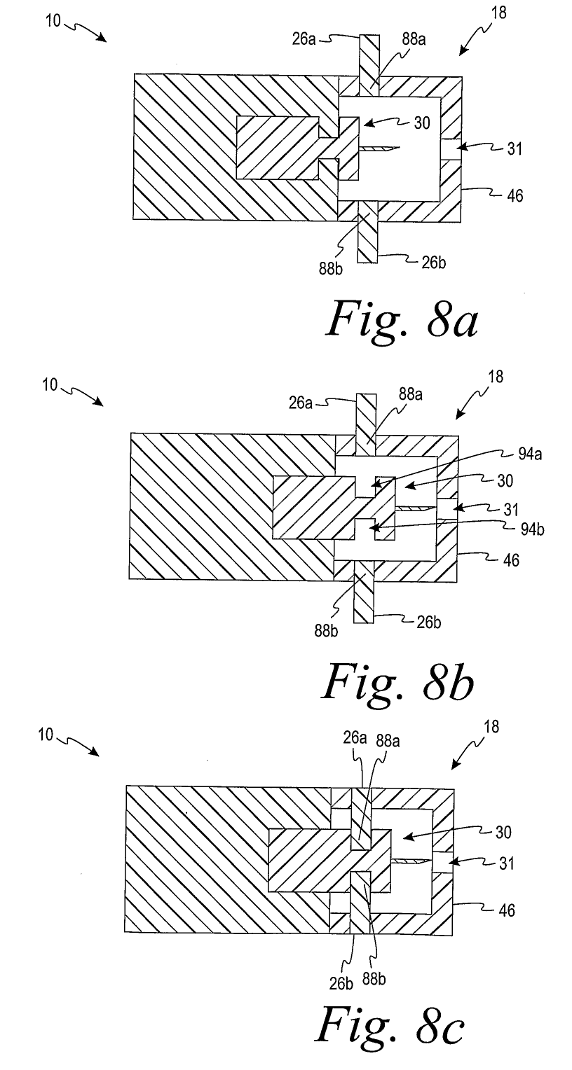

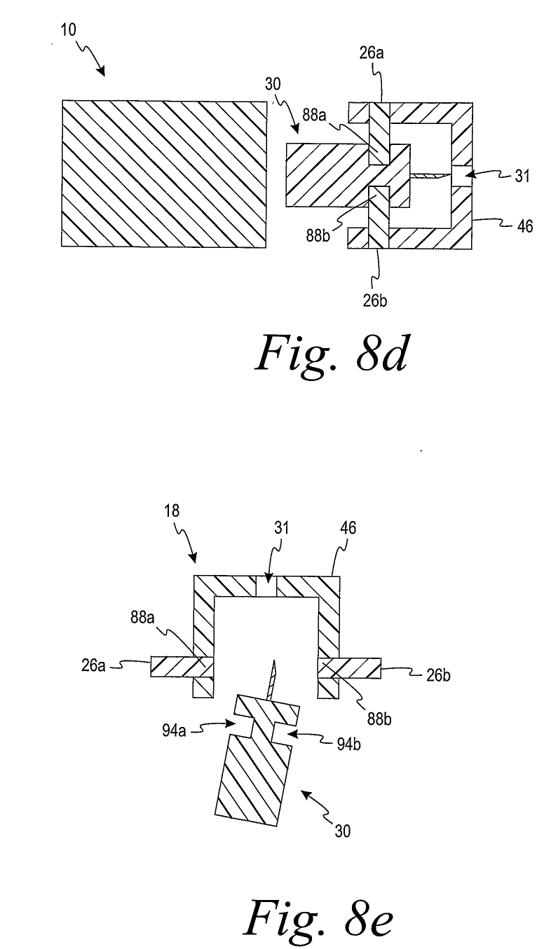

Referring also to FIG. 7, as the engagement mechanisms 88a-b move towards each other, they each engage a notch 94a-b formed on the lancet body 32 of the lancet assembly 30. A faceplate 96 is provided proximate the notches 94a-b between the notches 94a-b and the lance 34. As will be further detailed with respect to FIGS. 8a-8e, as the end cap 18 is removed from the lancing device 10 with the lancet-release mechanism 56 in the release position, the engagement mechanisms 88a-b engage the notches 94a-b and are prevented from disengaging the lancet assembly 30 by the faceplate 96.

Referring now to FIGS. 8a-e, the operation of the lancet-release mechanism 56 will be illustrated according to one embodiment of the present invention. FIG. 8a is a schematic representation of the lancing device 10 with the end cap 18 removably attached thereto. The lancet assembly 30 is illustrated in its cocked position. In this position, the engagement mechanisms 88a-b are separated from the notches 94a-b and the release buttons 26a-b extend from the external surface 28 (FIGS. 1a-b) of the end cap housing 46. FIG. 8b illustrates the lancet assembly 30 in its resting position. In this position, the notches 94a-b are aligned with the engagement mechanisms 88a-b but the engagement mechanisms 88a-b remain separate therefrom.

As illustrated in FIG. 8c, the release buttons 26a-b have been depressed thus forcing the engagement mechanisms 88a-b to engage the notches 94a-b of the lancet assembly 30. The end cap 18 is then removed as illustrated in FIG. 8d causing the lancet assembly 30 to release from the lancet holder 36 (FIGS. 3-5). As the end cap 18 is removed, the engagement mechanisms 88a-b apply a force on the lancet assembly 30 opposite the lancing device 10. This force is applied to the faceplate 96 (FIG. 7) that forms a surface (on the lance 34 side of the lancet body 32) for the notches 94a-b. After the end cap 18 has been removed, the release buttons 26a-b are released and the springs 92a-b (FIGS. 7a-b) cause the engagement mechanisms 88a-b to disengage the notches 94a-b. This releases the lancet assembly 30 from the end cap 18 as illustrated in FIG. 8e.

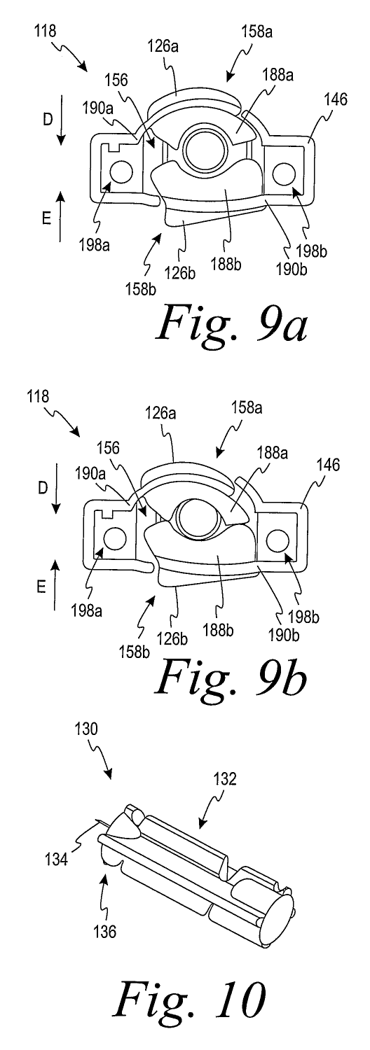

Turning now to FIGS. 9a-b, an end cap 118 is illustrated as viewed from the attachable lancing device 10, according to another embodiment of the present invention. The end cap 118 includes attachment areas 198a-b adapted to removably attach the end cap 118 to the support arms 20a-b of the lancing device 10. The end cap 118 includes a lancet-release mechanism 156 adapted to assist in the removal of the lancet assembly 130 (FIG. 10) from the lancet holder 36. The lancet-release mechanism 156 includes the release buttons 126a-b and a plurality of engagement mechanisms 188a-b. The release buttons 126a-b and engagement mechanisms 188a-b form cantilevers 158a-b that are adapted to move at pivot points 190a-b, respectively. The cantilevers 158a-b are integrally formed with the end cap housing 146.

As illustrated in FIG. 9b, when the release buttons 126a-b are depressed in the direction of the lancet assembly 130, illustrated by Arrows D and E in FIG. 9b, the engagement mechanisms 188a-b and release buttons 126a-b move at pivots 190a-b. This movement causes the engagement mechanisms 188a-b of the lancet-release mechanism 156 to engage the lancet assembly 136 as will be detailed further with respect to FIG. 10.

The engagement mechanisms 188a-b may be made of any material sufficient to grasp the lancet assembly 130 and release it from the lancet holder 36. For example, in one embodiment of the present invention, the engagement mechanisms 188a-b are made of rubber. In another embodiment of the present invention, the release buttons 126a-b and the engagement mechanisms 188a-b are made of rubber while the cantilevers 158a-b include a layer of plastic between the release buttons 126a-b and the engagement mechanisms 188a-b.

FIG. 10 illustrates a lancet assembly 130 according to yet another embodiment of the present invention. The lancet assembly 130 includes a lancet body 132 having a lance 134 extending therefrom. A head 136 is located at the end of the lancet body 132 proximate the lance 134. The head 136 is adapted to allow the engagement mechanisms 188a-b to engage the lancet assembly 130 and exert a force thereon sufficient to release the lancet assembly 130 from the lancet holder 36 (FIGS. 3-5).

### Alternative Embodiment A

An end cap removably attachable to a lancing device, the end cap comprising:

an end cap having an external surface;

a plurality of release buttons accessible from external the end cap housing; and

a plurality of engagement mechanisms, each of the plurality of engagement mechanisms being attached to one of the plurality of release buttons, the plurality of engagement mechanisms being adapted to engage a lancet assembly,

wherein the depression of the plurality of release buttons towards the external surface of the end cap housing causes the plurality of engagement mechanisms to engage the lancet assembly.

### Alternative Embodiment B

The end cap of Alternative Embodiment A wherein the plurality of engagement mechanisms is attached with at least one spring, the at least one spring being adapted to bias the engagement mechanisms into a resting position.

### Alternative Embodiment C

The end cap of Alternative Embodiment A wherein the lancet assembly includes at least one notch adapted to be engaged by one of the plurality of engagement mechanisms when the plurality of release buttons are depressed.

### Alternative Embodiment D

The end cap of Alternative Embodiment A wherein the plurality of release buttons extends through the end cap housing.

### Alternative Embodiment E

The end cap of Alternative Embodiment A wherein the plurality of release buttons and the plurality of engagement mechanisms form cantilevers.

### Alternative Embodiment F

The end cap of Alternative Embodiment E wherein the cantilevers move at a plurality of pivots when the plurality of release buttons are depressed, the movement of the cantilevers causing the engagement mechanisms to engage the lancet assembly.

### Alternative Embodiment G

A lancing device comprising:

a main housing;

a lancet holder located within the main housing, the lancet holder forming an aperture, the aperture being adapted to receive a lancet assembly therein; and

an end cap adapted to be removably attached to the main housing, the end cap having a plurality of release buttons accessible from external the end cap, each of the plurality of release buttons attached to an engagement mechanism adapted to engage the lancet assembly,

wherein the depression of the plurality of release buttons causes the plurality of engagement mechanisms to engage the lancet assembly such that removal of the end cap from the main housing removes the lancet assembly from the lancet holder.

### Alternative Embodiment H

The end cap of Alternative Embodiment G wherein the plurality of engagement mechanisms is attached with at least one spring, the at least one spring being adapted to bias the engagement mechanisms into a resting position.

### Alternative Embodiment I

The end cap of Alternative Embodiment G wherein the lancet assembly includes at least one notch adapted to be engaged by one of the plurality of engagement mechanisms when the plurality of release buttons are depressed.

### Alternative Embodiment J

The end cap of Alternative Embodiment G wherein the plurality of release buttons extends through the end cap housing.

### Alternative Embodiment K

The end cap of Alternative Embodiment G wherein the plurality of release buttons and the plurality of engagement mechanisms form cantilevers.

### Alternative Embodiment L

The end cap of Alternative Embodiment K wherein the cantilevers move at a plurality of pivots when the plurality of release buttons are depressed, the movement of the cantilevers causing the engagement mechanisms to engage the lancet assembly.

### Alternative Embodiment M

A method for removing a lancet assembly from a lancet holder, the method comprising the acts of:

providing a lancing device that includes the lancet holder located within a main housing, the lancet holder forming an aperture, the aperture being adapted to receive the lancet assembly therein;

providing an end cap adapted to be removably attached to the main housing, the end cap having a plurality of release buttons accessible from external the end cap, each of the plurality of release buttons being attached to an engagement mechanism adapted to engage the lancet assembly;

depressing at least one of the plurality of release buttons in the direction of the lancet assembly;

engaging the lancet assembly with the plurality of engagement mechanisms when at least one of the plurality of release buttons is depressed;

detaching the end cap from the main housing; and

removing the lancet assembly from the lancet holder as the end cap is detached from the main housing.

Alternative Process N

The method of Alternative Process M wherein the plurality of release buttons and the plurality of engagement mechanisms form cantilevers.

Alternative Process O

The method of Alternative Process N wherein the cantilevers move at a plurality of pivots when the plurality of release buttons are depressed, the movement of the cantilevers engaging the lancet assembly with the plurality of engagement mechanisms.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents and alternatives falling within the spirit and scope of the invention as defined by the appended claims.

1. An end cap removably attachable to a lancing device, the end cap comprising:
an end cap having an external surface;
a plurality of release buttons being accessible from external the end cap housing; and
a plurality of engagement mechanisms, each of the plurality of engagement mechanisms being attached to one of the plurality of release buttons, the plurality of engagement mechanisms being adapted to engage a lancet assembly,
wherein the depression of the plurality of release buttons towards the external surface of the end cap housing causes the plurality of engagement mechanisms to engage the lancet assembly.

2. The end cap of claim 1, wherein the plurality of engagement mechanisms is attached with at least one spring, the at least one spring being adapted to bias the engagement mechanisms into a resting position.

3. The end cap of claim 1, wherein the lancet assembly includes at least one notch adapted to be engaged by one of the plurality of engagement mechanisms when the plurality of release buttons are depressed.

4. The end cap of claim 1, wherein the plurality of release buttons extends through the end cap housing.

5. The end cap of claim 1, wherein the plurality of release buttons and the plurality of engagement mechanisms form cantilevers.

6. The end cap of claim 5, wherein the cantilevers move at a plurality of pivots when the plurality of release buttons are depressed, the movement of the cantilevers causing the engagement mechanisms to engage the lancet assembly.

7. A lancing device comprising:
a main housing;
a lancet holder located within the main housing, the lancet holder forming an aperture, the aperture being adapted to receive a lancet assembly therein; and
an end cap adapted to be removably attached to the main housing, the end cap having a plurality of release buttons accessible from external the end cap, each of the plurality of release buttons being attached to an engagement mechanism adapted to engage the lancet assembly,
wherein the depression of the plurality of release buttons causes the plurality of engagement mechanisms to engage the lancet assembly such that removal of the end cap from the main housing removes the lancet assembly from the lancet holder.

8. The end cap of claim 7, wherein the plurality of engagement mechanisms is attached with at least one spring, the at least one spring being adapted to bias the engagement mechanisms into a resting position.

9. The end cap of claim 7, wherein the lancet assembly includes at least one notch adapted to be engaged by one of the plurality of engagement mechanisms when the plurality of release buttons are depressed.

10. The end cap of claim 7, wherein the plurality of release buttons extends through the end cap housing.

11. The end cap of claim 7, wherein the plurality of release buttons and the plurality of engagement mechanisms form cantilevers.

12. The end cap of claim 11, wherein the cantilevers move at a plurality of pivots when the plurality of release buttons are depressed, the movement of the cantilevers causing the engagement mechanisms to engage the lancet assembly.

13. A method for removing a lancet assembly from a lancet holder, the method comprising the acts of:
providing a lancing device that includes the lancet holder located within a main housing, the lancet holder forming an aperture, the aperture being adapted to receive the lancet assembly therein;
providing an end cap adapted to be removably attached to the main housing, the end cap having a plurality of release buttons accessible from external the end cap, each of the plurality of release buttons being attached to an engagement mechanism adapted to engage the lancet assembly;
depressing at least one of the plurality of release buttons in the direction of the lancet assembly;
engaging the lancet assembly with the plurality of engagement mechanisms when at least one of the plurality of release buttons is depressed;
detaching the end cap from the main housing; and
removing the lancet assembly from the lancet holder as the end cap is detached from the main housing.

14. The method of claim 13, wherein the plurality of release buttons and the plurality of engagement mechanisms form cantilevers.

15. The method of claim 14, wherein the cantilevers move at a plurality of pivots when the plurality of release buttons are depressed, the movement of the cantilevers engaging the lancet assembly with the plurality of engagement mechanisms.
